(19) United States
(12) Patent Application Publication
(10) Pub. No.: US 2019/0200951 A1
(43) Pub. Date: Jul. 4, 2019

(54) NEEDLE TRACKING TRANSDUCER ARRAY METHODS AND APPARATUS

(71) Applicant: GLO-TIP, LLC, Redwood City, CA (US)

(72) Inventor: Joseph H. MEIER, McKinney, TX (US)

(21) Appl. No.: 16/317,529

(22) PCT Filed: Dec. 16, 2016
(86) PCT No.: PCT/US16/67325

§ 371 Date: Jan. 11, 2019

Related U.S. Application Data
(60) Provisional application No. 62/268,413, filed on Dec. 16, 2015.
(60) Provisional application No. 62/321,651, filed on Apr. 12, 2016.

(51) Int. Cl. A61B 8/08; A61B 5/06; A61B 8/00; A61B 5/00

(52) U.S. Cl. CPC A61B 8/0841; A61B 5/06; G06T 19/00; A61B 8/54; A61B 5/6848; A61B 8/5207

(57) ABSTRACT

Disclosed herein are systems and methods for providing real-time monitoring of a probe within a target zone. An apparatus for tracking the probe comprises a transducer assembly comprising a two-dimensional array of transducer elements. The two-dimensional array comprises a plurality of transverse arrays and a plurality of longitudinal arrays. The monitoring system further comprises a processor configured to activate and receive data from at least one transverse array extending along a transverse axis that is transverse to the target zone and to a direction of travel of the probe, and two or more longitudinal arrays extending along longitudinal axes that are transverse to the transverse axis. The two or more longitudinal arrays may be activated sequentially in a programmed sequence. Based on the data, the processor can determine the position of the probe within the target zone, and display the probe on a transverse cross-section view of the target zone via a software-generated special effect.

### CROSS-REFERENCE

This application claims the benefit of U.S. Provisional Patent Application Ser. No. 62/268,413, filed Dec. 16, 2015, entitled “NEEDLE TRACKING TRANSDUCER ARRAY METHODS AND APPARATUS” and U.S. Provisional Patent Application Ser. No. 62/321,651, filed Apr. 12, 2016, entitled “NEEDLE TRACKING TRANSDUCER ARRAY METHODS AND APPARATUS,” the entire disclosures of which are incorporated herein by reference. Additionally, the subject matter of the present application is related to U.S. application Ser. No. 14/703,708, filed May 4, 2015, entitled “HANDHELD IMAGING DEVICES AND RELATED METHODS”, the entire disclosure of which is incorporated herein by reference.

### BACKGROUND OF THE INVENTION

Non-invasive monitoring systems, such as ultrasound devices, can produce real-time images of blood vessels, organs, bones, nerves, tumors, and other target structures under the skin or other layers of tissue in patients. Such monitoring systems can be applied to aid procedures for interventional radiology, epidural placements, lumbar punctures, nerve blocks, tumor biopsies, and the cannulation of vascular vessels, among other procedures, by monitoring the position of a needle or probe with respect to the target zone. For example, the application of a non-invasive monitoring system to a vascular vessel cannulation procedure can help prevent unwanted results, such as the puncturing of wrong vascular vessels or structures, and/or repeated painful attempts to locate and cannulate the correct structure.

Prior methods and devices for non-invasive monitoring can be less than ideally suited for facilitating the insertion of a probe into a target zone of a patient. For example, in many prior monitoring systems, a two-dimensional tomographic image of the target zone displayed to the medical practitioner does not show the position of the probe tip in real time, requiring the practitioner to search the position of the probe tip. As a result, a high level of hand/eye coordination is required to perform the procedure, as the practitioner manipulates the probe with the hand while observing the tomographic images generated by the monitoring system.

In light of the above, it would be desirable to provide a monitoring system that can represent real-time internal images of the target zone and the position of the probe with respect to the target zone, thereby facilitating the performance of the procedure. Ideally, such a monitoring system is computationally efficient, cost-effective, and simple for a user to operate.

### Incorporation by Reference

All publications, patents, and patent applications mentioned in this specification are herein incorporated by reference to the same extent as if each individual publication, patent, or patent application was specifically and individually indicated to be incorporated by reference.

### SUMMARY

The methods and devices disclosed herein provide improved tracking of elongate probes inserted into a patient. Specifically, the methods and devices disclosed herein provide real-time tracking of the position of a probe with respect to a target zone in the tissue of the patient, using arrays of various orientations within a two-dimensional ultrasound transducer array to generate various cross-sections of the tissue. A processor operably coupled to the two-dimensional transducer array may be configured to activate one or more arrays in a programmed sequence to generate data relating to the position of the probe. Although reference is made the cannulation of a blood vessel, the methods and devices disclosed herein can be used to track elongate probes inserted into the tissue for many procedures, such as epidural placements, lumbar punctures, and nerve blockings.

In one aspect, an apparatus for facilitating intra-tissue inspection of a probe at a target zone comprises a transducer assembly and a processor. The transducer assembly comprises a two-dimensional array of transducer elements. The two-dimensional array comprises a plurality of transverse arrays and a plurality of longitudinal arrays. Each transverse array extends along a transverse axis of the two-dimensional array, and each longitudinal array extends along a longitudinal axis of the two-dimensional array that is transverse to the transverse axis. The processor is configured to activate at least one transverse array, wherein the at least one transverse array extends along a transverse axis that is transverse to the target zone and to a direction of travel of the probe. The processor is further configured to activate two or more longitudinal arrays sequentially in a programmed sequence. The processor is further configured to receive, from the at least one transverse array, data comprising a transverse cross-section of the target zone. The processor is further configured to receive, from the two or more longitudinal arrays, data comprising a longitudinal cross-section of at least a portion of the probe. The processor is further configured to determine, based on the data from the two or more longitudinal arrays, a position of a probe tip of the probe with respect to the two-dimensional array. The processor is further configured to generate a transverse cross-section view of the target zone based on the data from the at least one transverse array, the transverse cross-section view having depth coordinates and transverse coordinates, and the probe tip having a corresponding depth coordinate and transverse coordinate in the transverse cross-section view. The processor is further configured to display the transverse cross-section view with a probe indicator at the depth coordinate and transverse coordinate corresponding to the probe tip.

The processor may be further configured to select a longitudinal sampling window comprising a subset of the plurality of longitudinal arrays of the two-dimensional array. The subset may comprise one or more longitudinal arrays collectively configured to produce one or more longitudinal cross-sections of a complete length of the probe. The processor may be configured to selectively activate the one or more longitudinal arrays of the longitudinal sampling window. The processor may be further configured to adjust a width of the longitudinal sampling window or selection of the subset of the plurality of longitudinal arrays comprising the longitudinal sampling window based on a position or orientation of the probe.

The processor may be further configured to select a subset of the plurality of longitudinal arrays of the two-dimensional array for use in determination of the position of the probe tip. The subset may comprise one or more longitudinal arrays collectively configured to produce one or more longitudinal cross-sections of a complete length of the probe.

The two-dimensional array may further comprise one or more diagonal arrays extending along a diagonal axis that is oriented at an oblique angle to the transverse axis. The processor may be further configured to activate the one or more diagonal arrays and receive from the one or more diagonal arrays data comprising a diagonal cross-section of at least a portion of the probe. The one or more diagonal arrays may comprise two or more diagonal arrays, and the processor may be configured to activate the two or more diagonal arrays sequentially in a programmed sequence.

The at least one transverse array and the two or more longitudinal arrays may be activated simultaneously at similar frequencies. The at least one transverse array and the two or more longitudinal arrays may be activated simultaneously at different frequencies. The at least one transverse array and the two or more longitudinal arrays may be activated simultaneously at substantially non-interfering frequencies.

The two or more longitudinal arrays may comprise all of the plurality of longitudinal arrays of the two-dimensional array. The processor may be configured to activate the plurality of longitudinal arrays in a programmed sequence to sample all transducer elements of the two-dimensional array.

The processor may be configured to determine the position of the probe tip at predetermined time intervals, and update the display of the transverse cross-section of the target zone at each time interval to show the probe indicator at depth and transverse coordinates corresponding to the position of the probe tip determined at each time interval. The predetermined time intervals may substantially match a rate of data acquisition by the programmed sequence of the two or more longitudinal arrays. The predetermined time intervals may substantially match a rate of data acquisition by each activated transverse array or longitudinal array.

All transducer elements of a single activated transverse array or longitudinal array may be pulsed simultaneously. Transducer elements of a single activated transverse array or longitudinal arrays may each be pulsed individually in a timed sequence. The processor may be configured to generate a three-dimensional image of the target zone and the probe based on the data received from the at least one transverse array and the two or more longitudinal arrays.

The probe indicator may comprise one or more symbols or shapes displayed using one or more colors, animations, or other software-generated special effects.

The processor may be further configured to determine a projected probe path of the probe based on the position of the probe tip at two or more time points. The processor may be further configured to display the transverse cross-section view with a projected probe trajectory at depth and transverse coordinates corresponding to the projected probe path. One of the two or more time points may be an insertion time point of insertion of the probe into the target zone, wherein the probe tip is at a known, predetermined position at the insertion time point. The projected probe trajectory may comprise one or more of a colorized line, dashed line, dotted line, flashing line, or an arrow.

The processor may be further configured to determine a position, with respect to the two-dimensional array, of a target location within the target zone. The processor may be further configured to display the transverse cross-section view with a target hit indicator at depth and transverse coordinates corresponding to the probe tip when the position of the target location matches the position of the probe tip. The target hit indicator may comprise one or more of a radiating or glowing tip of the probe indicator, a flashing tip of the probe indicator, or a color change of a tip of the probe indicator.

The processor may be further configured to generate and display a topographical rendition of the target zone based on the data from the at least one transverse array or the two or more longitudinal arrays.

The processor may be further configured to identify one or more tissue structures of the target zone in the displayed transverse cross-section view. The processor may be configured to identify the one or more tissue structures based on one or more of a shape, density, relative position, pulsatility, or echogenicity of the one or more tissue structures as determined with the data from the at least one transverse array or the two or more longitudinal arrays.

In another aspect, a method for providing real-time monitoring of a probe at a target zone comprises positioning a transducer assembly over the target zone, the transducer assembly comprising a two-dimensional array of transducer elements having a plurality of transverse arrays and a plurality of longitudinal arrays. The method further comprises activating at least one transverse array, wherein the at least one transverse array extends along a transverse axis that is transverse to the target zone and to a direction of travel of the probe. The method further comprises activating two or more longitudinal arrays sequentially in a programmed sequence, wherein each longitudinal array extends along a longitudinal axis that is transverse to the transverse axis. The method further comprises obtaining, from the at least one transverse array, data comprising a transverse cross-section of the target zone. The method further comprises obtaining, from the two or more longitudinal arrays, data comprising a longitudinal cross-section of at least a portion of the probe. The method further comprises determining, based on the data from the two or more longitudinal arrays, a position of a probe tip of the probe with respect to the two-dimensional array. The method further comprises generating a transverse cross-section view of the target zone based on the data from the at least one transverse array, the transverse cross-section view having depth coordinates and transverse coordinates, and the probe tip having a corresponding depth coordinate and transverse coordinate in the transverse cross-section view. The method further comprises displaying the transverse cross-section view with a probe indicator at the depth coordinate and transverse coordinate corresponding to the probe tip.

The method may further comprise selecting a longitudinal sampling window comprising a subset of the plurality of longitudinal arrays of the two-dimensional array. The subset may comprise one or more longitudinal arrays collectively configured to produce one or more longitudinal cross-sections of a complete length of the probe. Activating two or more longitudinal arrays may comprise selectively activating the one or more longitudinal arrays of the longitudinal sampling window. The method may further comprise adjusting a width of the longitudinal sampling window or selection of the subset of the plurality of longitudinal arrays comprising the longitudinal sampling window based on a position or orientation of the probe.

The method may further comprise selecting a subset of the plurality of longitudinal arrays comprising one or more longitudinal arrays collectively configured to produce one or more longitudinal cross-sections of a complete length of the probe. The determining may comprise determining the position of the probe tip based on data from the subset of the plurality of longitudinal arrays.

The two-dimensional array may further comprise one or more diagonal arrays extending along a diagonal axis that is oriented at an oblique angle to the transverse axis. The method may further comprise activating the one or more diagonal arrays and receiving from the one or more diagonal arrays data comprising a diagonal cross-section of at least a portion of the probe. The one or more diagonal arrays may comprise two or more diagonal arrays, and the two or more diagonal arrays may be activated sequentially in a programmed sequence.

The at least one transverse array and the two or more longitudinal arrays may be activated simultaneously at similar frequencies. The at least one transverse array and the two or more longitudinal arrays may be activated simultaneously at different frequencies. The at least one transverse array and the two or more longitudinal arrays may be activated simultaneously at substantially non-interfering frequencies.

The two or more longitudinal arrays may comprise all of the plurality of longitudinal arrays of the two-dimensional array, and activating the two or more longitudinal arrays may comprise activating the plurality of longitudinal arrays in a programmed sequence to sample all transducer elements of the two-dimensional array.

The determining may comprise determining the position of the probe tip is at predetermined time intervals. The displaying may comprise updating the transverse cross-section of the target zone at each time interval to show the probe indicator at depth and transverse coordinates corresponding to the position of the probe tip determined at each time interval. The predetermined time intervals may substantially match a rate of data acquisition by the programmed sequence of the two or more longitudinal arrays. The predetermined time intervals may substantially match a rate of data acquisition by each activated transverse array or longitudinal array.

All transducer elements of a single activated transverse array or longitudinal array may be pulsed simultaneously. Transducer elements of a single activated transverse array or longitudinal arrays may each be pulsed individually in a timed sequence. The method may further comprise generating and displaying a three-dimensional image of the target zone and the probe based on the data received from the at least one transverse array and the two or more longitudinal arrays.

The probe indicator may comprise one or more symbols or shapes displayed using one or more colors, animations, or other software-generated special effects.

The method may further comprise determining a projected probe path of the probe based on the position of the probe tip at two or more time points, and displaying the transverse cross-section view with a projected probe trajectory at depth and transverse coordinates corresponding to the projected probe path. One of the two or more time points may be an insertion time point of insertion of the probe into the target zone, wherein the probe tip is at a known, predetermined position at the insertion time point. The projected probe trajectory may comprise one or more of a colorized line, dashed line, dotted line, flashing line, or an arrow.

The method may further comprise determining a position, with respect to the two-dimensional array, of a target location within the target zone. The method may further comprise displaying the transverse cross-section view with a target hit indicator at depth and transverse coordinates corresponding to the probe tip when the position of the target location matches the position of the probe tip. The target hit indicator may comprise one or more of a radiating or glowing tip of the probe indicator, a flashing tip of the probe indicator, or a color change of a tip of the probe indicator.

In another aspect, an apparatus for facilitating intra-tissue inspection of a probe at a target zone comprises a transducer assembly and a processor, the transducer assembly comprising a two-dimensional array of transducer elements, and the two-dimensional array comprising a plurality of longitudinal arrays. The processor is configured to activate the plurality of longitudinal arrays sequentially in a programmed sequence and receive, from the plurality of longitudinal arrays, data comprising a plurality of longitudinal cross-sections of the probe. The processor is further configured to determine, based on the data from the plurality of longitudinal arrays, a position and an orientation of the probe with respect to the two-dimensional array. The processor is further configured to select, based on the position and orientation of the probe, a longitudinal sampling window comprising a subset of the plurality of longitudinal arrays. The subset may comprise one or more longitudinal arrays collectively configured to produce one or more longitudinal cross-sections of the probe over a complete length of the probe. The processor is further configured to activate the one or more longitudinal arrays of the longitudinal sampling window sequentially in a programmed sequence.

The processor may be further configured to adjust a width of the longitudinal sampling window or selection of the subset of the plurality of longitudinal arrays of the longitudinal sampling window, based on a position and orientation of the probe.

The processor of any embodiment disclosed herein may be configured with instructions to define the window so as to correspond to a first area of the array with dimensions sized smaller than a second area of the two-dimensional array. Circuitry coupled to the array may be configured to sample data over the second area sized larger than the first area.

In any embodiment disclosed herein, the window may correspond to a portion of the array and the processor may be configured with instructions to sample in hardware only a portion of the array defined with the window.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic diagram of an exemplary monitoring system.

FIG. 2A shows a transducer assembly suitable for incorporation with a monitoring system as disclosed herein.

FIGS. 2B and 2C show exemplary configurations of transverse arrays of the transducer assembly of FIG. 2A.

FIGS. 2D and 2E show exemplary configurations of longitudinal arrays of the transducer assembly of FIG. 2A.

FIG. 2F shows an exemplary configuration of a diagonal array of the transducer assembly of FIG. 2A.

FIG. 2G shows exemplary diagonal arrays of a transducer assembly as disclosed herein.

FIG. 3 illustrates the three-dimensional scanning of a target zone using phased two-dimensional transducer arrays.

FIGS. 4A-4D schematically illustrate the tracking of a probe using a transducer assembly as disclosed herein.

FIGS. 5A-5C show exemplary displayed images generated by the monitoring system of FIGS. 4A-4B over a time course.

FIGS. 6A-6C show exemplary displayed images generated by the monitoring system of FIGS. 4C-4D over a time course.

FIGS. 7A and 7B illustrate the projection of a travel path of a probe using a monitoring system as disclosed herein.

FIGS. 8A and 8B illustrate the display of a target hit indicator using a monitoring system as disclosed herein.

FIG. 9 shows a flowchart of a method for providing real-time monitoring of a probe at a target zone.

FIG. 10 shows a flowchart of a method 1000 for providing real-time monitoring of a probe at a target zone.

### DETAILED DESCRIPTION OF THE INVENTION

Disclosed herein are methods, systems, and devices for non-invasive ultrasound imaging of a target zone in a patient. In particular, disclosed herein are methods and devices for monitoring the insertion of a probe into the tissue of the patient, and indicating to a user the real-time position of the probe with respect to the target zone within the tissue.

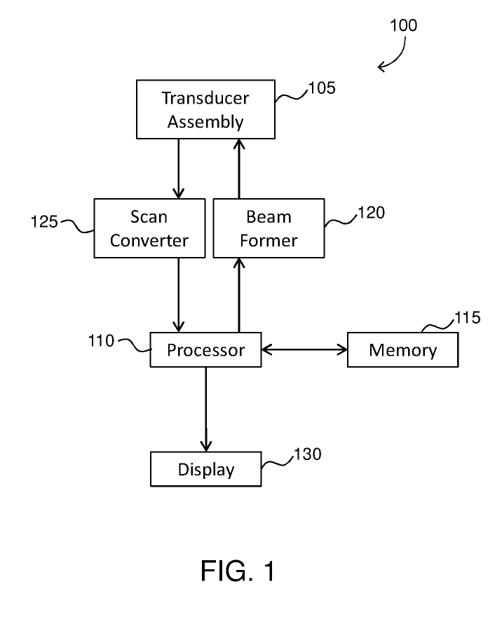

FIG. 1 is a schematic diagram of an exemplary monitoring system 100. The system comprises a transducer assembly 105 and a processor 110, wherein the processor is configured to control the operation of the transducer assembly. The system may further comprise a memory 115, a beamformer 120, and a scan converter 125, operatively coupled to the processor 110. The memory 115 may be configured to store software instructions for operating the monitoring system. The beamformer 120 may be configured to receive instructions from the processor to control operation of the transducer assembly. For example, the beamformer may be configured to control one or more of the timing, strength, angle, amplitude, and phase of ultrasound signals transmitted by the transducer assembly. The beamformer can comprise at least one of a B-mode, F-mode, and a D-mode acquisition mode. The scan converter 125 may be configured to receive scan data from the transducer assembly, typically comprising ultrasound signals received by the transducer assembly, and convert the scan data into an image format. The image data may be transmitted to the processor, wherein the processor may in turn transmit the image data to a display 130 coupled to the processor 110. The display may be configured to display one or more images of the tissue region scanned by the transducer assembly to a user of the monitoring system.

The processor 110 may comprise a microprocessor such as a general microprocessor for personal computers, and/or a specialized microprocessor for a specific implementation such as analog and mixed signal operations. The memory 115 may store software instructions for operating the monitoring system, and/or information such as images scanned using the monitoring system. The memory may comprise non-volatile memory, such as flash memory, and/or magnetic storage such as hard disks. The memory may comprise a removable memory device, such as Secure Digital (SD) cards. The processor 110 and memory 115 may be combined to form a microcontroller.

The display may be a stand-alone display device operatively coupled to the monitoring system. Alternatively or in combination, the display may be an integrated display provided with the transducer assembly. For example, the transducer assembly, processor, memory, and display may be enclosed in a housing to provide a single, integrated hand-held imaging device. Additional details regarding configurations of a hand-held imaging device may be found in copending U.S. patent application Ser. No. 14/703,708 and U.S. Pat. No. 9,022,940, the entire contents of which are incorporated herein by reference.

The monitoring system 100 can be used in the medical field for intra-tissue or sub-dermal inspection of a patient. For example, the monitoring system may be used to facilitate the non-invasive imaging of vascular vessels, such as veins and arteries, through skin and/or other tissue. In one example, such imaging can be useful in guiding a medical practitioner performing a vascular vessel cannulation procedure, allowing the medical practitioner to align, position, and guide a probe such as a needle or a catheter into the vascular vessel.

The monitoring system will be described herein primarily in relation to the cannulation of a vessel using a probe such as needle. However, one of skill in the art will appreciate that this is not intended to be limiting, and the devices and methods disclosed herein may be used in other applications involving the monitoring of a moving object within a medium. For example, the monitoring system may be used to identify specific structures (e.g., imperfections) in a material to guide the insertion of an object into the material.

While the transducer assembly is described herein primarily as a two-dimensional transducer array, the transducer assembly may alternatively comprise a three-dimensional transducer array, the three-dimensional transducer array comprising one or more two-dimensional arrays as described herein stacked vertically, or in the z-axis direction. The three-dimensional transducer array may be operated substantially as described herein with respect to a two-dimensional transducer array, wherein two or more layers of the two-dimensional arrays may be activated simultaneously or in a programmed sequence.

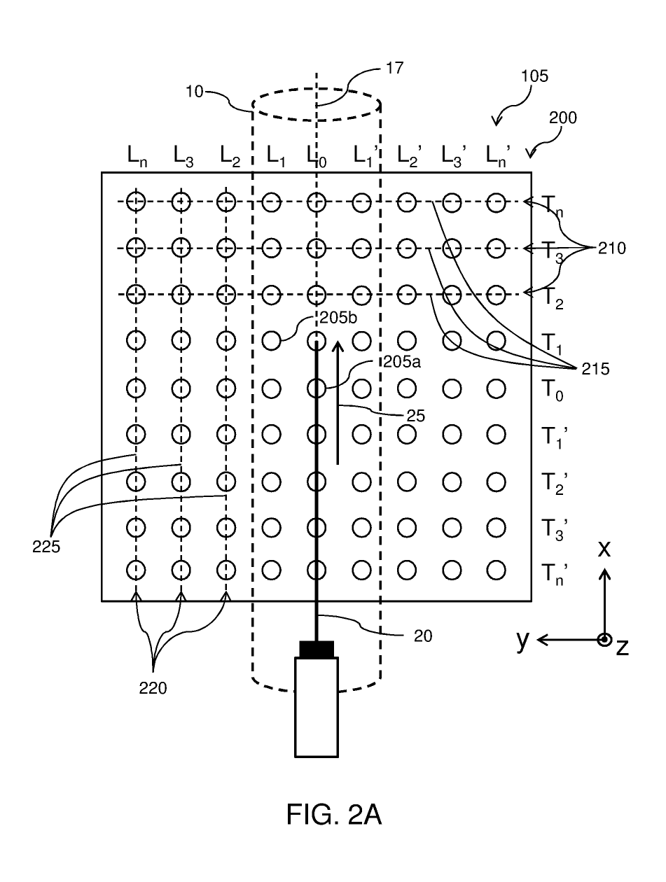

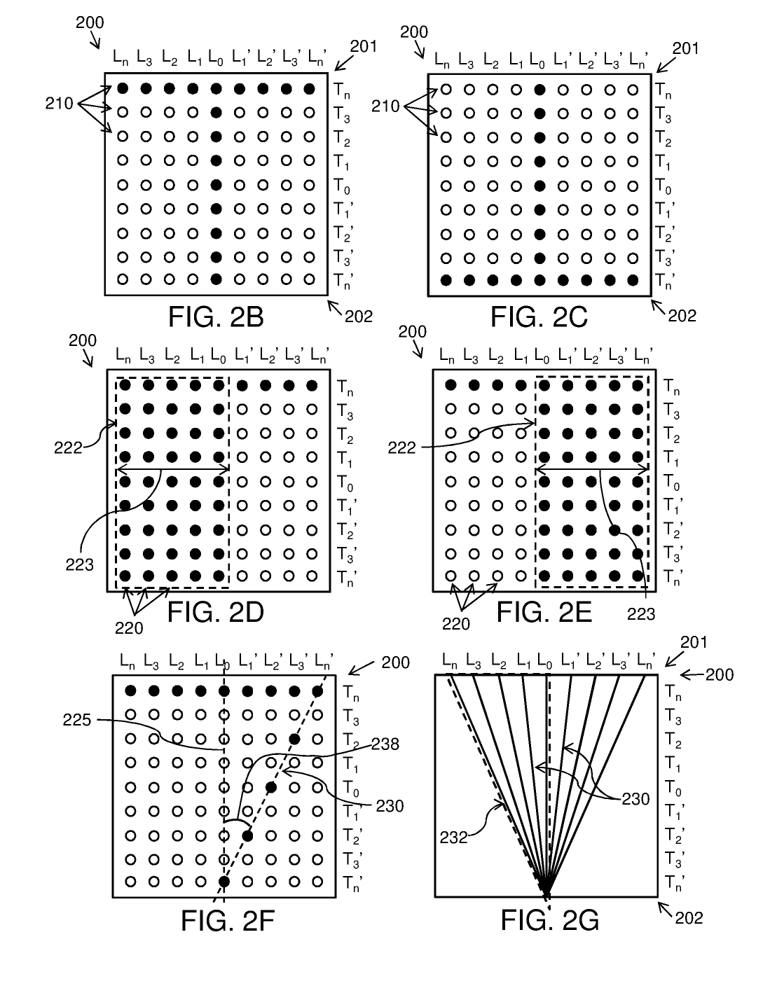

FIG. 2A shows a transducer assembly 105 suitable for incorporation with a monitoring system as disclosed herein. The transducer assembly 105 comprises a plurality of transducer elements 205, such as piezoelectric elements, configured to emit beams of ultrasound energy and to detect reflections of ultrasonic beams. The transducer elements may be arranged in a two-dimensional array 200 as shown. The two-dimensional array may comprise a plurality of transverse arrays 210, each labeled Tₙ-Tₙ′, and a plurality of longitudinal arrays 215, each labeled Lₙ-Lₙ′. Each transverse array may be formed by a plurality of transducer elements aligned or extending along a transverse axis 220 of the two-dimensional array, wherein the transverse axis may extend along a width of the transducer assembly or be parallel to the y-axis of the transducer assembly as shown in FIG. 2A. Each longitudinal array may be formed by a plurality of transducer elements aligned or extending along a longitudinal axis 225 of the two-dimensional array that is transverse to a transverse axis 220, wherein the longitudinal axis may extend along a length of the transducer assembly or be parallel to x-axis of the transducer assembly as shown in FIG. 2A. Each transducer element of the two-dimensional array may have a known position on the two-dimensional array with respect to the longitudinal and transverse arrays. For example, transducer element 205a may be positioned on longitudinal array L₀ and on transverse array T₀, whereas transducer element 205b may be positioned on longitudinal array L₁ and on transverse array T₁. A transverse array and a longitudinal array may or may not partially overlap. For example, as shown in FIG. 2A, transverse array Tₙ may overlap with longitudinal array Lₙ at the transducer element Lₙ/Tₙ, wherein the transducer element Lₙ/Tₙ is a part of both the transverse array Tₙ and the longitudinal array Lₙ.

In operation, the transducer assembly 105 may be placed over the tissue of the patient containing the target zone 10, and oriented such that one or more transverse arrays are positioned transversely to the target zone and to a direction of travel of the probe. For example, as shown in FIG. 2A, the target zone may comprise a blood vessel for cannulation, and the transducer assembly 105 may be placed over the skin of the patient above the blood vessel such the transverse arrays 210 are transverse to the longitudinal axis 17 of the target zone as shown. When activated, the transverse arrays can sample a transverse cross-section of the target zone, as described in further detail herein. A probe 20 may be inserted into the patient's tissue towards the blood vessel, such that the direction of travel 25 of the probe is transverse to the transverse arrays. One or more longitudinal arrays of the transducer assembly can be activated to track the probe along its length as the probe is navigated towards the target zone, as described in further detail herein.

FIGS. 2B and 2C show exemplary configurations of transverse arrays of the transducer assembly 105 of FIG. 2A. One or more transverse arrays 210 of the two-dimensional transducer array 200 of the transducer assembly may be activated by a processor operably coupled to the transducer assembly (e.g., processor 110 shown in FIG. 1) to obtain data of one or more transverse cross-sections of the target zone. FIG. 2B shows the two-dimensional array 200 with an activated transverse array Tₙ, positioned near the distal end 201 of the two-dimensional array. FIG. 2C shows the transducer assembly 200 with an activated transverse array Tₙ′, positioned near the proximal end 202 of the two-dimensional array. As shown, the processor may selectively activate any transverse array at any position within the two-dimensional array. The selection of the transverse array for activation may be fixed to provide a stable view of the transverse cross-section of the target zone, wherein the user may adjust the position of the transverse array with respect to the target zone by manually moving the transducer assembly. For example, the processor may be configured to sample transverse array Tₙ to obtain a stable view of the transverse cross-section of the target zone near the distal end of the two-dimensional array, and the user may move the transducer assembly to align the transverse array Tₙ over a different cross-section of the target zone as desired. Alternatively, the selection of the transverse array for activation may be adjustable by the user of the monitoring system, such that the position of the transverse array with respect to the target zone may be adjusted without requiring manual movement of the transducer assembly. For example, the user may select transverse array Tₙ′ for activation, to obtain a transverse cross-section of the target zone near the proximal end of the two-dimensional array. While FIGS. 2B and 2C show the activation of only single transverse arrays, the processor may activate any number of transverse arrays as appropriate for obtaining sufficient data for the generation of a transverse cross-sectional image of the target zone. For example, the processor may activate two or more transverse arrays, and data from the two or more transverse arrays may be used to produce the transverse cross-sectional image of the target zone.

FIGS. 2D and 2E show exemplary configurations of longitudinal arrays of the transducer assembly 105 of FIG. 2A. One or more longitudinal arrays 220 of the two-dimensional transducer array 200 of the transducer assembly may be activated by a processor operably coupled to the transducer assembly (e.g., processor 110 shown in FIG. 1) to obtain data of one or more longitudinal cross-sections of the target zone, including data of the position of the probe with respect to the two-dimensional array. FIG. 2D shows the two-dimensional array 200 with five activated longitudinal arrays L₀-Lₙ. FIG. 2E shows the transducer assembly 200 with five activated longitudinal arrays L₀-Lₙ′. As shown, the processor may selectively activate any longitudinal array at any position within the two-dimensional array. While FIGS. 2D and 2E show the activation of five longitudinal arrays, the processor may activate any number of longitudinal arrays as appropriate for obtaining sufficient data for the determination of probe position with respect to the two-dimensional array. The processor may selectively activate a subset of the longitudinal arrays, the subset comprising a plurality of adjacent longitudinal arrays forming a longitudinal sampling window 222 having a width 223. For example, FIG. 2D shows a longitudinal sampling window 222 comprising longitudinal arrays L₀-Lₙ, while FIG. 2E shows a longitudinal sampling window 222 comprising longitudinal arrays L₀-Lₙ′. In both of the configurations shown in FIGS. 2D and 2E, the width 223 of the longitudinal sampling window corresponds to a width spanning five longitudinal arrays. The processing unit may select and dynamically adjust a longitudinal sampling window and its width based on the position and orientation of the probe, as described in further detail herein.

FIG. 2F shows an exemplary configurations of a diagonal array 230 of the transducer assembly 105 of FIG. 2A. In addition to transverse and longitudinal arrays, the two-dimensional transducer array 200 of the transducer assembly may also comprise one or more diagonal arrays 230, wherein a diagonal array comprises a plurality of transducer elements aligned or extending along a diagonal axis 235 at an oblique angle 238 to a longitudinal axis 225 of the two-dimensional array. One or more diagonal arrays of the transducer assembly may be activated by a processor operably coupled to the transducer assembly (e.g., processor 110 shown in FIG. 1) to obtain data of one or more diagonal cross-sections of the target zone, including data of the position of the probe with respect to the two-dimensional array. FIG. 2F shows the transducer assembly with an activated diagonal array comprising the transducer elements at L₀/Tₙ′, L₁/T₂′, L₂′/T₀, L₃′/T₂, and Lₙ′/Tₙ. FIG. 2G shows exemplary diagonal arrays 230 of a transducer assembly as disclosed herein. A probe inserted into the tissue at a position corresponding to L₀/Tₙ′ at the proximal end 202 of the two-dimensional array 200 may be tracked by activating one or more diagonal arrays. For example, diagonal arrays extending from L₀/Tₙ′ to various longitudinal positions along the transverse axis at the distal end 201 of the two-dimensional array may be activated, such as diagonal arrays extending between L₀/Tₙ′ and Lₙ/Tₙ, L₀/Tₙ′ and L₀/Tₙ, L₀/Tₙ′, L₀/Tₙ′ and Lₙ′/Tₙ, etc. The diagonal arrays may be activated sequentially in a programmed sequence. The processor may selectively activate any diagonal array comprising transducer elements at any positions within the two-dimensional array. The processor may activate any number of diagonal arrays as appropriate for obtaining sufficient data for the determination of probe position with respect to the transducer assembly. The processor may selectively activate a plurality of adjacent diagonal arrays forming a diagonal sampling window 232. For example, as shown in FIG. 2G, a diagonal sampling window 232 may comprise the diagonal arrays extending between L₀/Tₙ′ and Lₙ/Tₙ, L₀/Tₙ′ and L₃/Tₙ, L₀/Tₙ′, L₀/Tₙ′ and L₂/Tₙ, L₀/Tₙ′, L₀/Tₙ′ and L₁/Tₙ′ and L₀/Tₙ′, L₀/Tₙ′ and L₀/Tₙ. The processing unit may select and dynamically adjust a diagonal sampling window (e.g., the number and orientation of diagonal arrays included in the sampling window) based on the position and orientation of the probe, as described in further detail herein.

The activated array configurations of FIGS. 2B-2G are shown and described by way of example only, and the two-dimensional array of transducer elements may be sampled in any other suitable array configuration or pattern to sample any desired cross section of the tissue near the target zone. The processor operably coupled to the transducer assembly (e.g., processor 110 shown in FIG. 1) may be configured to activate a plurality of transducer elements of the 2-dimensional array in any suitable pattern to sample the desired cross-sections of the tissue. For example, a longitudinal array or a transverse array may be partially sampled, such that the activated longitudinal or transverse array extends over only a portion of the width or length of the two-dimensional array. A diagonal array may extend diagonally in any axis or orientation with respect to the two-dimensional array. Although only arrays activated in linear patterns are shown in FIGS. 2B-2F, the processor may be configured to activate an array of transducer elements having a non-linear pattern, such as an array having a curvilinear pattern or any other suitable pattern or shape, extending in any suitable orientation over the two-dimensional array.

A plurality of arrays of the two-dimensional transducer array, such as one or more of a transverse array, a longitudinal array, and a diagonal array, may be operated at similar frequencies or at different frequencies. A plurality of arrays may be operated at frequencies that are substantially non-interfering with one another, such that different transducer arrays may be operated simultaneously to concurrently obtain data of different cross-sections of the target zone.

A plurality of arrays of the two-dimensional array may be oscillated on and off, simultaneously or in a programmed, timed sequence. A plurality of arrays may be oscillated on and off at various rates, sequences, or patterns to sample the overall grid. The oscillation sequence of the arrays may be a function of software programming (e.g., stored in the memory 115 and executed by the processor 110), and may be configured to provide continuous surveillance of the entire two-dimensional array. For example, referring to FIG. 2A, all or a portion of the plurality of longitudinal arrays of the two-dimensional array may be continuously sampled in a programmed oscillation sequence (e.g., all of the arrays from Lₙ to Lₙ′, or a portion of the arrays from Lₙ to L₀). For example, longitudinal arrays of a select longitudinal sampling window may be continuously sampled in a programmed oscillation sequence. Alternatively or additionally, all or a portion of the plurality of transverse arrays of the two-dimensional array may be continuously sampled in a programmed sequence (e.g., all of the arrays from Tₙ to Tₙ′, or a portion of the arrays from Tₙ to T₀). Each of the plurality of arrays activated in a programmed sequence may be alternatingly oscillated relative to one another, such that when a first array is activated for scanning along a first axis, a second array is deactivated to cease scanning along a second axis, and when the second array is activated for scanning along the second axis, the first array is deactivated to cease scanning along the first axis. Preferably, the transducer arrays are oscillated on and off at a suitable rate to maintain data acquisition and fluid image display. For example, the rate of data acquisition by each transducer array may be in a range from about 24 hertz (Hz) to about 38 kHz, or the rate of data acquisition by each programmed oscillation sequence of a plurality of arrays may be in a range from about 24 Hz to about 38 kHz. The one or more transducer arrays may be configured to acquire data at a sufficient rate to enable image display at a frame rate of at least 24 frames per second (fps), or a frame rate within a range from about 24 fps to about 38,000 fps, wherein higher rates of data acquisition may be used to support a higher frame-rate image display for the monitoring of faster-moving objects, for example in robotic surgery applications. The system may be configured to have a latency within a range from about 50 ms to about 1 ms from the oscillation of a transducer array to the update of the displayed image based on the acquired data.

One or more arrays of the two-dimensional array may not oscillate on and off and instead scan the target zone continuously, in order to continuously sample a select cross-section of the target zone. For example, as described in further detail herein, a transverse array of the two-dimensional array may be configured to continuously scan the corresponding transverse cross-section of the tissue, to provide a fixed view of a target zone of interest. Optionally, two or more transverse arrays may be configured to continuously scan the corresponding transverse cross-sections of the tissue, and information from the two or more cross-sections may be collectively processed to provide the fixed view of the target zone. Each continuously sampled transducer array may be configured to acquire data at a rate of about 24 hertz (Hz) to about 38 kHz, or at a sufficient rate to enable image display at a frame rate of at least 24 frames per second (fps), or a frame rate within a range from about 24 fps to about 38,000 fps.

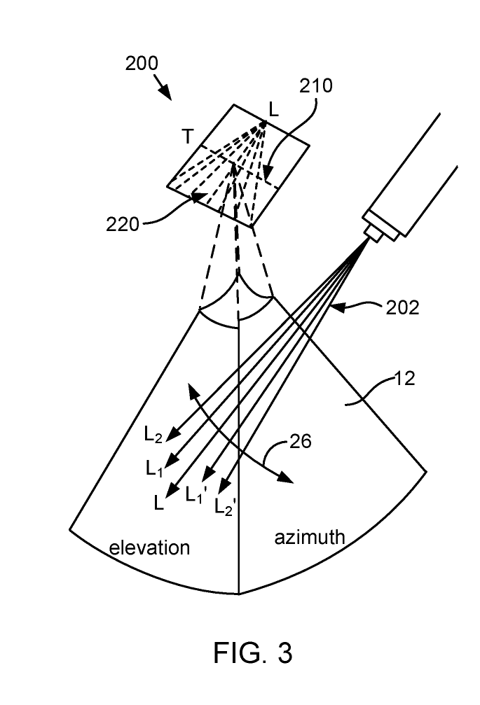

FIG. 3 illustrates the three-dimensional scanning of a target zone using phased two-dimensional transducer arrays. A two-dimensional ultrasound transducer array 200 as disclosed herein may comprise a plurality of transverse arrays 220, longitudinal arrays 210, and/or diagonal arrays, each of which may comprise a linear array or a phased array. A linear array can comprise transducer elements that are activated simultaneously together to generate a transverse cross-sectional image of the target zone. A phased array can comprise transducer elements that are each pulsed individually in a timed sequence. The two-dimensional array may comprise a combination of linear and phased arrays, which together can generate data relating to the target zone as well as the depth, length, and lateral movement 26 of the probe 20 in real time. Optionally, the entire two-dimensional array may be operated as a phased array to generate data relating the three-dimensional volume 12 of the scanned target zone. The two-dimensional phased array may be operated using focusing and steering techniques known in the art to produce image data of the three-dimensional volume of tissue.

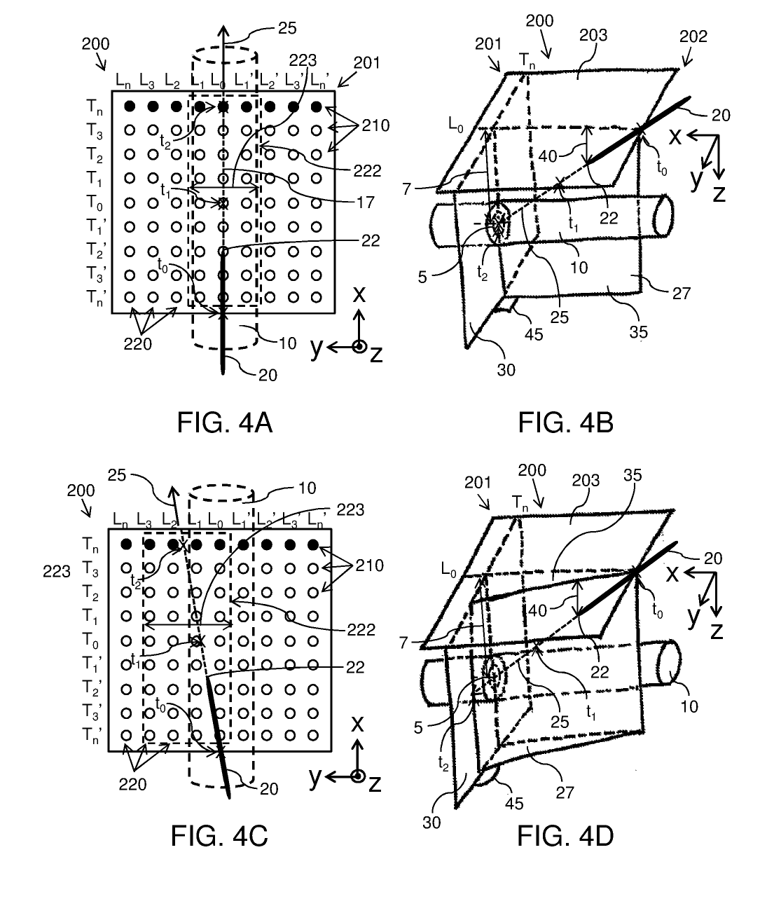

FIGS. 4A-4D schematically illustrate the tracking of a probe using a transducer assembly as disclosed herein. FIGS. 4A and 4C are top views and FIGS. 4B and 4D are side perspective views of the transducer assembly 105 tracking the probe 20 as it is inserted into the tissue of the patient and navigated towards a target location 5 within a target zone 10, such as a blood vessel. The target location 5 can correspond to the intended final position of the probe within the target zone. As described herein, the two-dimensional transducer array 200 of the transducer assembly may comprise one or more transverse arrays 210 configured to sample one or more transverse cross-sections 30 of the target zone that are transverse to the longitudinal axis 17 target zone and/or the direction of travel 25 of the probe. A plurality of transverse arrays may be oscillated on and off in a programmed sequence, to scan a plurality of transverse cross-sections of the tissue that partially or completely span the two-dimensional array. Alternatively or additionally, one or more transverse arrays may be continuously sampled to provide data for the image display of the target zone comprising the target location. For example, as shown in FIGS. 4A-4D, the transducer assembly may be positioned over the target zone to align the distal transverse array Tₙ over the target location 5 at a depth 7 from the plane 203 of the two-dimensional transducer array, and the transverse array Tₙ located near the distal end 201 of the two-dimensional array may be sampled continuously to obtain an image of the transverse cross-section 30 of the target zone containing the target location 5.

When the probe is located within a scanning range of a transverse array, the transverse cross-section corresponding to the transverse array may contain at least a portion of the probe. In such cases, the width of the probe may be determined from the transverse cross-section scan containing the probe. When the probe is located outside of the scanning range of the transverse array, the corresponding transverse cross-sections of the target zone will not contain the probe. Accordingly, to track the probe as it travels towards the target zone, one or more longitudinal arrays 220 may be sampled as described herein, wherein the one or more longitudinal cross-sections 35 of the target zone scanned by the longitudinal arrays may contain at least a portion of the probe. For example, a longitudinal sampling window including longitudinal arrays Lₙ-Lₙ′ and having a width corresponding to the width spanning the longitudinal arrays Lₙ-Lₙ′ may be sampled in a programmed sequence while the probe is being navigated towards the target zone, to capture the corresponding longitudinal cross-sections of the target zone containing the probe. The transducer assembly may be positioned over the target zone with the distal transverse array Tₙ and the central longitudinal array L₀ positioned over the target location 5 of the target zone, such that the target location 5 is positioned at a depth 7 from the L₀/Tₙ position of the two-dimensional array. Such positioning of the transducer assembly can optimize tracking and visualization of the location of the probe with respect to the target location, as described in further detail herein. The data from the longitudinal arrays can also be used to measure the depth of the probe with respect to the plane 203 of the two-dimensional transducer array 220. For example, the depth 40 of the probe tip 22, or the vertical distance between the probe tip and the plane of the two-dimensional transducer array, may be determined based on the time between the transmission of an ultrasound signal and the sensing of a reflected ultrasound signal by the transducer array.

To improve the efficiency of data acquisition and processing, only a subset or portion of the longitudinal and/or diagonal arrays of the two-dimensional array may be sampled, wherein the scans generated by the subset of arrays collectively contain the complete length of the probe. The processor may be configured to select the longitudinal and/or diagonal sampling window comprising one or more adjacent longitudinal and/or diagonal arrays whose cross-sectional scans collectively contain the complete length of the probe. The position and orientation of the probe at one or more time points may be determined based on one or more initial scans using the entire two-dimensional array. Based on the known shape of the probe, and its position and orientation at one or more time points, a projected travel path of the probe with respect to the two-dimensional array may be determined, as described in further detail herein. An appropriate longitudinal and/or diagonal sampling window may then be selected based on the current position and orientation of the probe, and/or based on the projected path of the probe. For example, the sampling window may be selected to include the subset of arrays whose scanning range the entire length of the probe is currently positioned in, or the sampling window may be selected to include the subset of arrays whose scanning range the entire length of the probe is positioned in throughout the complete projected travel path of the probe. The position and orientation of the probe may be determined at a plurality of time points during the probe's travel, and the processor may dynamically adjust the selection of the sampling windows based on the current position, orientation, and/or projected path of the probe. For example, the width of a longitudinal sampling window may be adjusted, and/or the selection of the subset of arrays of the sampling window may be adjusted. Such selective sampling of the two-dimensional transducer array can not only enable faster and more efficient data capture by omitting scans with arrays that do not contain useful information (e.g, do not contain the probe), but can also reduce computational burden on the system since the amount of data to be processed and analyzed is greatly reduced compared to continuous scans with the entire two-dimensional array.

Alternatively, to improve the efficiency of data processing, only data from a subset or portion of the longitudinal and/or diagonal arrays containing the length of the probe may be analyzed. For example, the plurality of longitudinal arrays of the two-dimensional array may be sampled continuously throughout the travel of the probe, and the processor may determine which of the longitudinal arrays contain the complete length of the probe, as described herein in reference to selection of a longitudinal sampling window (e.g., based whether a longitudinal scan from a given longitudinal array contains an ultrasound signal reflected from the probe). Subsequently, only data from the portion of the longitudinal arrays containing the length of the probe may be processed further to determine the position and orientation of the probe with respect to the two-dimensional array as described in further detail herein. The processor may be configured to dynamically adjust the selection of the arrays from which data is processed, based on the position and orientation of the probe throughout travel. The window of the array can be defined in hardware or software, and combinations thereof. For example, the entire array can be sampled in hardware with data acquisition and the windowed defined in software so as to comprise only a portion of the sampled data array. Alternatively or in combination, the processor may comprise circuitry to activate only a portion of the array corresponding to the window, and to capture data from only the portion of the window. In both instances, such selective data processing can reduce computational burden, enabling faster and more efficient data analysis as well as improving the efficiency of power consumption by the system.

While the window and sampling can be configured in many ways with hardware and software, the processor can be configured with instructions to define the window so as to correspond to a first area of the array with dimensions sized smaller than a second area of the two-dimensional array, and the circuitry coupled to the array can be configured to sample data over the second area sized larger than the first area. Alternatively or in combination, the window may correspond to a portion of the array, and the processor can be configured with instructions to sample in hardware only a portion of the array defined with the window.

As shown in FIGS. 4A and 4B, in some cases, the probe may approach the target zone with the longitudinal plane 27 of the probe oriented orthogonally to the plane of the transverse cross-section 30 of the target zone imaged by the transverse array, such that the angle 45 between the longitudinal plane 27 of the probe and the plane of the transverse cross-section 30 of the target zone is about 90°. In such cases, the longitudinal plane 27 of the probe may overlap with a longitudinal cross-section 35 of the tissue obtained by one or more longitudinal arrays 220, such that one or more longitudinal cross-sections may contain the entire length of the probe. For example, if the probe is oriented with its longitudinal axis 17 aligned with longitudinal array L₀, as shown in FIGS. 4A and 4B, the longitudinal cross-section obtained with the longitudinal array L₀ can contain the complete length of the probe. To ensure capture of data containing the length of the probe while reducing power consumption by the system and computational burden in data processing, a longitudinal sampling window 222 comprising longitudinal arrays L₁, L₀, and L₁′ may be sampled continuously in a programmed sequence. Alternatively, the sampling window 222 may comprise only the longitudinal array L₀, as long the longitudinal axis 17 of the probe remains in alignment with the longitudinal array L₀ such that the array L₀ can capture data comprising the entire length of the probe. The position, orientation, and projected path of the probe may be determined at a plurality of time points during travel of the probe, and the longitudinal sampling window may be adjusted accordingly. For example, if a change is detected in the orientation of the probe, such that its longitudinal axis 17 is no longer aligned with longitudinal array L₀, the width 223 of the longitudinal sampling window 222 may be increased and/or a different set of longitudinal arrays may be selected for sampling to ensure capture of the complete length of the probe.

In some cases, as shown in FIGS. 4C and 4D, the probe may approach the target zone with the longitudinal plane 27 of the probe oriented at an oblique angle 45 to the plane of the transverse cross-section 30 of the target zone. In such cases, the longitudinal plane 27 of the probe does not overlap completely with a single longitudinal cross-section 35 of the tissue obtained by a longitudinal array, but can partially overlap with a plurality of longitudinal cross-sections obtained by a plurality of longitudinal arrays. In the example shown in FIGS. 4C and 4D, the longitudinal plane 27 of the probe can partially overlap with the longitudinal cross-sections obtained by longitudinal arrays L₃, L₂, L₁, and L₀. To ensure that the probe is detected in the scan data, a plurality of longitudinal arrays, such as all or a portion of the longitudinal arrays of the two-dimensional array, may be sampled in a programmed sequence. To ensure capture of data containing the length of the probe while reducing power consumption by the system and computational burden in data processing, a longitudinal sampling window 222 comprising longitudinal arrays L₃, L₂, L₁, and L₀ may be sampled continuously in a programmed sequence. The position, orientation, and projected path of the probe may be determined at a plurality of time points during travel of the probe, and the longitudinal sampling window may be adjusted accordingly. For example, if a change is detected in the orientation of the probe, such that the angle 45 between the longitudinal plane 27 of the probe and the plane of the transverse cross-section 30 of the target zone changes, the width 223 of the longitudinal sampling window 222 may be increased and/or a different set of longitudinal arrays may be selected for sampling to ensure capture of the complete length of the probe. Additionally or alternatively to sampling a plurality of longitudinal arrays, one or more diagonal arrays may be sampled to scan the length of the probe. For example, for a probe oriented as shown in FIGS. 4C and 4D, the processor may be configured to sample one or more diagonal arrays comprising a plurality of transducer elements aligned or extending along or substantially parallel to the longitudinal axis 17 of the probe. A plurality of diagonal arrays may be sampled in a programmed sequence to sample the length of the probe. To ensure capture of data containing the length of the probe while reducing power consumption by the system and computational burden in data processing, a diagonal sampling window comprising only a portion of diagonal arrays of the two-dimensional array may be sampled continuously in a programmed sequence.

Referring again to FIGS. 4A-4D, the various arrays within the two-dimensional array may be sampled over time as the probe is inserted into the tissue and navigated towards the target location 5 within the target zone 10, in order to track the depth, width, and length of the probe within the tissue in real time. Various cross-sections of the tissue may be scanned at a plurality of time points during throughout the insertion and navigation of the probe, wherein the transducer array may be configured to generate the cross-sections at predetermined time intervals. For example, as shown in FIGS. 4A and 4B, the volume of tissue below the two-dimensional transducer array may be scanned at times tₒ, t₁, and t₂, wherein the probe is inserted into the tissue at time t₀, the probe is between the insertion point and the plane of the transverse-cross section 30 at time t₁, and the probe has reached the plane of the transverse-cross section 30 at time t₂.

The real-time position of the probe respect to the target zone may be displayed to the user for visual monitoring of probe progression. The image displayed to the user may comprise image data generated by one or more various arrays of the transducer assembly. For example, the displayed image may comprise a transverse cross-section of the target zone, a longitudinal cross-section of the target zone, or a cross-section of the target zone along any other axis of the two-dimensional transducer array (such as a diagonal cross-section). The cross-sectional view of the target zone may be a fixed view of a specific region of the target zone, or the cross-sectional view may be a dynamically changing view of cross-sections of the tissue as the probe travels towards the target zone. Alternatively or additionally, the displayed image may comprise a three-dimensional view of the entire volume of tissue scanned by the two-dimensional transducer array.

The displayed image may further comprise an image of the probe overlaid on the cross-section view of the target zone. For example, the image of the probe may comprise an image of a transverse cross-section of the probe (showing the width of the probe), a longitudinal cross-section of the probe (showing the length of the probe), or a view of the probe from any side of the three-dimensional volume of tissue scanned by the two-dimensional array (such as a top view or a side view of the probe). The displayed image, showing both the target zone and the real-time position of the probe within the tissue, can be refreshed at a rate that is suitable for providing a substantially real-time view of probe position. For example, the displayed image may be refreshed at a rate that substantially matches the rate of data acquisition by the two-dimensional array, the rate of data acquisition by each activated transducer array, and/or the rate of data acquisition by a single programmed oscillation sequence of a plurality of transverse or longitudinal arrays. Preferably, the image is refreshed at a rate that is undetectable to the user viewing the displayed image, such that a fluid image display is maintained.

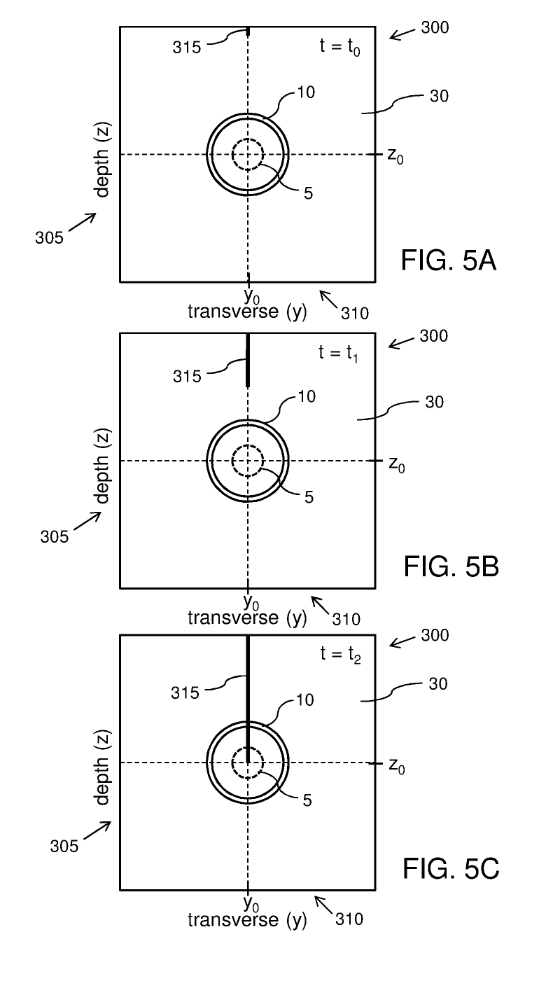

FIGS. 5A-5C show exemplary displayed images generated by the monitoring system of FIGS. 4A-4B over a time course. The displayed image 300 comprises a view of the transverse cross-section 30 of the target zone 10 generated by a transverse array, wherein the target zone comprises a blood vessel and the transverse cross-section is transverse to the longitudinal axis of the blood vessel. The transverse cross-section view of the target zone is displayed with respect to a plurality of depth (z) coordinates 305 and a plurality of transverse (y) coordinates 310, wherein the depth coordinates correspond to the distance of the displayed objects from the plane of the two-dimensional transducer array, and the transverse coordinates correspond to the position of the displayed objects along the transverse axis of the two-dimensional transducer array. For example, the central transverse coordinate y₀ may correspond to the position along the distal transverse array Tₙ corresponding to the central longitudinal array L₀. The displayed images may show the transverse cross-section 30 centered about the target location 5, to facilitate user tracking of the probe with respect to the target location. For example, as described herein, the transducer assembly may be positioned over the target zone to align the intersection of the distal transverse array Tₙ and the central longitudinal array L₀ over the target location 5, wherein the target location 5 is located at a depth 7 from the plane 203 of the two-dimensional transducer array. In this case, the displayed image may comprise the transverse cross-section view generated by the distal transverse array Tₙ, the z₀ coordinate may correspond to the depth 7, and the y₀ coordinate may correspond to the position of the central longitudinal array L₀. along the distal transverse array Tₙ.

The displayed image further comprises a probe indicator 315, overlaid on the image of the transverse cross-section 30 of the target zone. Specifically, a probe indicator may be displayed over the image of the target zone cross-section at depth and transverse coordinates corresponding to the real-time position of the probe tip. The probe indicator can be shown over the image of the transverse cross-section even when the probe is located outside the scanning range of the transverse array generating the transverse cross-section, via a software-generated special effect. As described herein, the processor may be configured to determine the depth of the probe with respect to the plane of the two-dimensional transducer array and the position of the probe with respect to the longitudinal and transverse arrays of the two-dimensional transducer array, based on data from one or more scans with longitudinal and/or diagonal arrays. The processor may be further configured to calculate the depth and transverse coordinates corresponding to the probe position. The probe indicator may be displayed at the one or more depth and transverse coordinates of the image corresponding to the position of the probe. The probe indicator can be continuously updated based on live data gathered from the various longitudinal and diagonal arrays of the two-dimensional transducer array, such that the substantially real-time position of the probe with respect to the target zone can be displayed to the user. For example, the processor may be configured to determine the spatial position of the probe at predetermined time intervals throughout the insertion and navigation of the probe in the tissue, calculate the corresponding depth and transverse coordinates at each time interval, and update the position of the probe indicator in the displayed image at each time interval.

FIG. 5A shows the displayed image 300 at time t₀, corresponding to time t₀ indicated in FIGS. 4A and 4B, when the probe 20 is inserted into the tissue of the patient. The probe indicator 315 is displayed at depth and transverse coordinates corresponding to the position of the probe at time t₀. FIG. 5B shows the displayed image 300 at time t₁, corresponding to time t₁ indicated in FIGS. 4A and 4B, when the probe is between the insertion point and the target location 5. The probe indicator 315 comprises a line extending along coordinates of the image corresponding to the position of the probe body at time t₁. FIG. 5C shows the displayed image 300 at time t₂, corresponding to time t₂ indicated in FIGS. 4A and 4B, when the probe has reached the target location 5. The probe indicator 315 comprises a line extending along coordinates of the image corresponding to the position of the probe body at time t₂. In FIGS. 5A-5C, the displayed images show the probe traveling along a path (aligned with longitudinal plane 27 of the probe 27, FIG. 4B) that crosses the target location 5, such that the probe eventually hits the target location at time t₂.

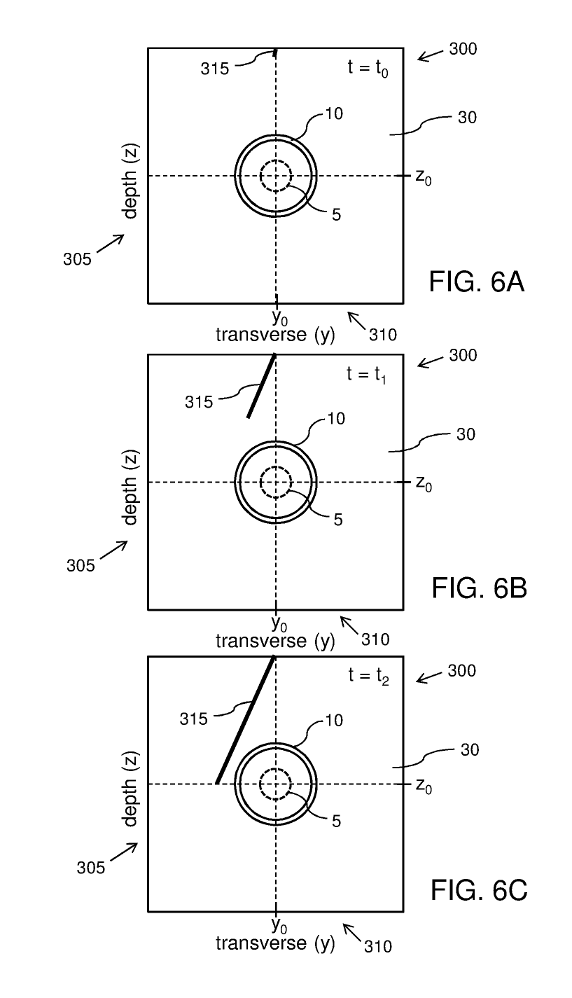

FIGS. 6A-6C show exemplary displayed images generated by the monitoring system of FIGS. 4C-4D over a time course. The displayed image 300 comprises a view of the transverse cross-section 30 of the target zone 10 generated by a transverse array, wherein the transverse cross-section view of the target zone is displayed with respect to a plurality of depth (z) coordinates 305 and a plurality of transverse (y) coordinates 310 as described herein. The transverse cross-section 30 is displayed centered about the target location 5, corresponding to the intended final position of the probe within the target zone, to facilitate user tracking of the probe with respect to the target location. The displayed image further comprises a probe indicator 315 substantially as described in reference to FIGS. 5A-5C. FIG. 6A shows the displayed image 300 at time t₀, corresponding to time t₀ indicated in FIGS. 4C and 4D, when the probe 20 is inserted into the tissue of the patient. FIG. 6B shows the displayed image 300 at time t₁, corresponding to time t₁ indicated in FIGS. 4C and 4D, when the probe is between the insertion point and the plane of the transverse cross-section 30. FIG. 6C shows the displayed image 300 at time t₂, corresponding to time t₂ indicated in FIGS. 4C and 4D, when the probe has reached the plane of the transverse cross-section 30. In FIGS. 6A-6C, the displayed images show the probe traveling along a path (aligned with longitudinal plane of the probe 27, as shown in FIG. 4D) that does not cross the target location 5, such that the probe misses the target location.

The displayed image may indicate the position of the probe at a plurality of time points during the movement of the probe (e.g., image may include indicators for the probe tip position at the different time points, each labeled with the corresponding time). Alternatively, the displayed image may simply indicate the current position of the probe, regardless of the time point. At any given time during the movement of the probe, the current position of the probe tip may be determined based on the most recent data generated from one or more transducer arrays. Assuming that the insertion location of the probe is known (e.g., position corresponding to L₀/Tₙ′ and at zero-depth from the surface of the two-dimensional transducer array (z=0)), the probe indicator can comprise a line extending between the position on the displayed image corresponding to the insertion location and the position on the displayed image corresponding to the current probe tip location.

The probe indicator of FIGS. 5A-6C is shown and described by way of example only, and the probe indicator may comprise any form appropriate for showing the real-time position of the probe. The probe indicator may comprise any symbol (e.g., dot, circle, square, cross, start, etc.) displayed at the depth and transverse coordinates corresponding to the position of the probe tip, wherein the symbol may move continuously in real time as the probe tip progresses towards the target location. Alternatively or additionally, the probe indicator may comprise any shape (e.g., solid line, dashed line, dotted line, etc.) extending over a plurality of depth and transverse coordinates corresponding to positions of multiple points along the probe body, which may include the probe tip. For example, the probe indicator may comprise a solid line showing the position of the probe body, and a differently colored dot corresponding to the probe tip. The probe indicator may be displayed using any suitable special effects, such as various colors or animations (e.g., flashing off and on, glowing or radiating, etc.).

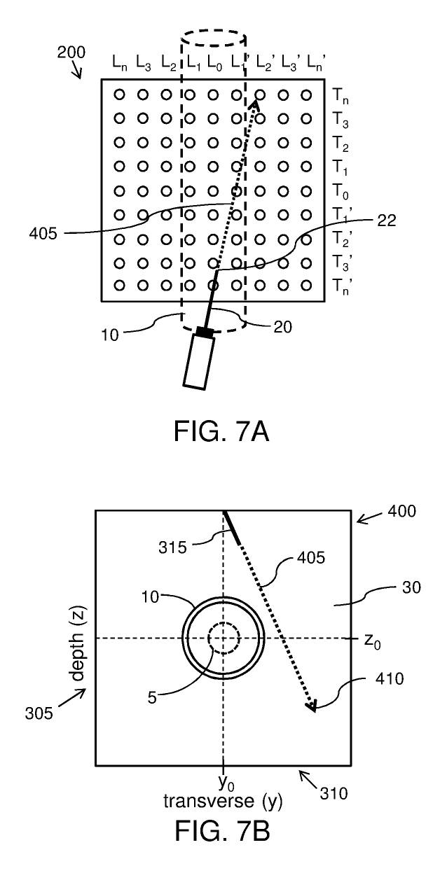

FIGS. 7A and 7B illustrate the projection of a travel path of a probe using a monitoring system as disclosed herein. FIG. 7A shows a projected path 405 of a probe 20 with respect to a two-dimensional transducer array 200 of a transducer assembly 105 as described herein. Using the sampling of the various arrays of the two-dimensional array over a time course of travel of the probe within the target zone 10 of the tissue, a projected path 405 or trajectory of the probe can be predicted. For example, the processor operatively coupled to the transducer assembly may be configured to determine the position of the probe tip 22 at two or more different time points, based on the scan data generated by one or more transducer arrays. Assuming that the probe does not bend to any significant degree and remains substantially linear during travel in the tissue, and therefore that the travel path of the probe is substantially linear, the projected path of the probe at future time points can be calculated based on the linear relationship between the positions of the probe tip at two or more different time points. One of the two different time points may be the insertion point of the probe into the tissue, at which the position of the probe tip may be known if the probe is inserted at a known location with respect to the transducer array. For example, if the probe is inserted into the tissue at the location corresponding to L₀/Tₙ′, the position of the probe tip at the insertion time point may be assumed to correspond to the L₀/Tₙ′ at z=0. In this case, the projected probe path may be calculated using the known, predetermined probe tip position at insertion, and the probe tip position determined at a time point during the travel of the probe.

FIG. 7B shows an exemplary displayed image 400 generated by the monitoring system, showing the projected probe path 405. The displayed image 400 can comprise a view of a transverse cross-section 30 of the target zone 10 and a probe indicator 315 as described herein, shown with respect to a plurality of depth (z) coordinates 305 and a plurality of transverse (y) coordinates 310 and preferably centered around the target location 5 of the target zone. The displayed image 400 can further comprise the projected probe trajectory 405 corresponding to the projected probe path determined as described herein. The projected probe trajectory can be overlaid on the image of the transverse cross-section via a software-generated special effect. For example, the projected probe trajectory can comprise a colorized line, dashed line, dotted line, flashing line, or any other chosen effect. The projected probe trajectory may comprise a directional indicator 410, such as an arrow head, to indicate the direction of probe travel. The projected probe trajectory can be re-calculated and updated in the display in real-time, based on the latest scan data of the target zone. The display of the projected probe path can enable the user to evaluate the chosen trajectory of the probe and the potential success in hitting the target location 5 of the target zone. In the example shown in FIGS. 7A and 7B, the display shows that the projected probe path does not cross the target location, informing the user that the direction of probe insertion should be adjusted in order for the probe to successfully reach the target location.

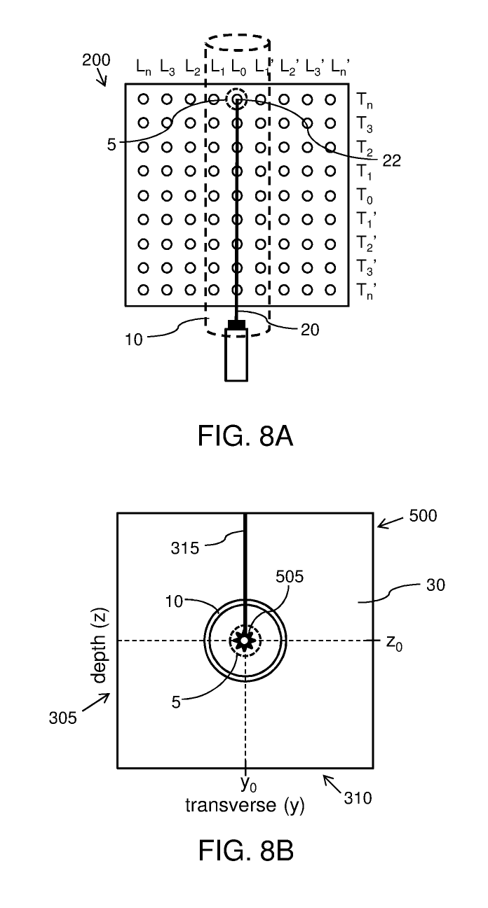

FIGS. 8A and 8B illustrate the display of a target hit indicator using a monitoring system as disclosed herein. FIG. 8A shows the tracking of a probe 20 navigated towards a target location 5 within a target zone 10, using a two-dimensional transducer array 200 of a transducer assembly 105 as described herein. A user of the monitoring system may position the two-dimensional transducer array over the patient's skin such that the distal transverse array Tₙ and the central longitudinal array L₀ are positioned above the target location 5, using the displayed image of the transverse cross-section of the target zone to adjust the placement of the transducer assembly. The target location may then be assumed to be positioned at a certain depth below the Tₙ/L₀ position of the two-dimensional transducer array. The processor may determine the depth based on data relating to the target zone generated by one or more transducer arrays. Alternatively or in combination, the processor may determine the position of the target location based on user guidance, wherein the user selects the target location from the displayed image of the transverse cross-section of the target zone (e.g., by touching a location on a touch-screen display, dragging a cursor to the desired location, etc.). The processor can then convert the coordinate position of the selected target location into the spatial position of the target location with respect to the two-dimensional transducer array. The distal transverse array Tₙ may be sampled continuously without moving the transducer assembly, in order to obtain a continuous image of the transverse cross-section 30 of the target zone 10 containing the target location 5.

FIG. 8B illustrates an exemplary displayed image comprising a target hit indicator, generated by the monitoring system of FIG. 8A. As described herein, the displayed image 500 may comprise an image of the transverse cross-section 30 of the target zone 10 containing the target location 5, as well as an overlaid image of a probe indicator 315 indicating the real-time position of the probe. The displayed image 500 may further comprise a target hit indicator 505 to visually indicate that the probe has reached the target location 5 within the target zone. The target hit indicator can be displayed when the processor determines that the spatial position of the probe tip substantially matches or is within a predetermined range of the spatial position of the target location. For example, assuming that the transducer assembly has been positioned with its central longitudinal array L₀ and distal transverse array Tₙ centered over the target location, the processor can determine that the probe tip has reached the target when the probe tip reaches a location that is positioned at the interface of the longitudinal cross-section generated by the central longitudinal array L₀ and the transverse array 30 generated by the distal transverse array Tₙ. The target hit indicator may comprise a special effect applied to the tip of the probe indicator, such as a radiating or glowing tip, a flashing tip, a color change of the tip, or any other suitable graphical effect. Optionally, the target location 5 may be highlighted in the image 500 via any suitable visual effect (dot, circle, cross, etc.), or the displayed image may be configured to be centered about the target location, such that the center of the displayed image is substantially aligned with the target location.

Optionally, the two-dimensional array may be further configured to generate a topographical rendition of the tissue for display through the monitoring system. For example, at least a portion of the transducer arrays may be configured to map the target tissue based on one or more of a shape of the tissue, the density of the tissue, the relative position of the tissue with respect to the array, the pulsatility of the tissue, or the echogenicity of the tissue. Based on the data, the processor coupled to the transducer arrays may be configured to recognize one or more structures or organs of the tissue, such as a blood vessel, a vein, an artery, or tissue masses. The identified tissue structures may be indicated on the displayed image, such as the transverse cross-sectional image of the target zones as described herein. For example, different tissue structures may be indicated to the user, for labeled with text or colors and combinations thereof In a blood vessel, for example, fluid inside the vessel may be displayed in black, while the more echogenic vessel wall may be displayed in a lighter color. The labeling can be appropriate for a user to distinguish an artery from a vein, for example by coloring arteries red and veins blue in order to ensure that the probe is inserted into the correct vessel.

Optionally, all or a portion of the transducer elements of the two-dimensional array may be adapted to perform Doppler ultrasound, wherein high-frequency ultrasound waves are emitted towards red blood cells and the reflections from the moving red blood cells are processed to measure blood flow and blood pressure. The measured ultrasound signals may be processed to obtain a Doppler frequency and produce a flow display or a Doppler sonogram.

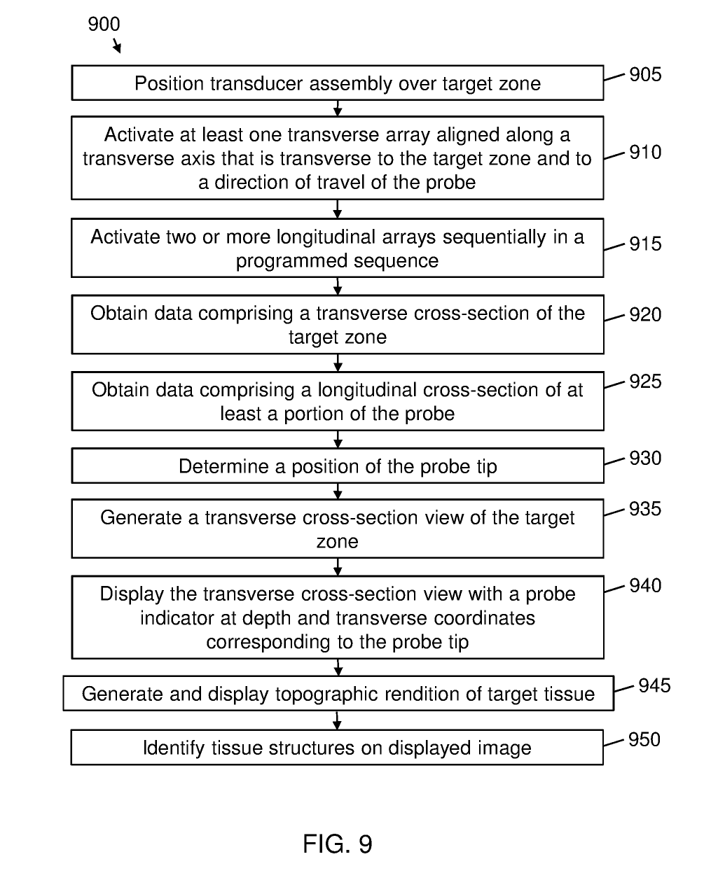

FIG. 9 shows a flowchart of a method 900 for providing real-time monitoring of a probe at a target zone.

In step 905, a transducer assembly is positioned over the target zone, wherein the transducer assembly comprises a two-dimensional transducer array as described herein. The transducer assembly may be placed such that the distal transverse array and central longitudinal array of the two-dimensional array are centered over a target location in the target zone, for example.

In step 910, at least one transverse array is activated, wherein the transverse array extends along a transverse axis that is transverse to the target zone and to a direction of travel of the probe. The at least one transverse array may comprise, for example, a distal transverse array that extends along a transverse axis of the target zone containing the target location, wherein the distal transverse array may be configured to continuously scan the transverse cross-section.

In step 915, two or more longitudinal arrays are activated sequentially in a programmed sequence, wherein each longitudinal array extends along a longitudinal axis that is transverse to the transverse axis. Additionally or alternatively to the two or more longitudinal arrays, two or more diagonal arrays aligned or extending along an axis at an oblique angle to a longitudinal axis of the two-dimensional array may be activated in a programmed sequence.

In step 920, data comprising a transverse cross-section of the target zone is obtained from the at least one transverse array. This data may be sent from the transverse array to a processor operably coupled thereto, wherein the processor may be configured to control the operation of the two-dimensional transducer array and/or receive and process the data generated using the transducer array.

In step 925, data comprising a longitudinal cross-section of at least a portion of the probe is obtained from the two or more longitudinal arrays. This data may be sent from the longitudinal arrays to the processor operably coupled to the transducer array.

In step 930, the position of the probe tip is determined based on the data from the two or more longitudinal arrays.

In step 935, a transverse cross-section view of the target zone is generated based on the data from the at least one transverse array. The transverse cross-section view comprises depth coordinates and transverse coordinate. The probe tip, whose position with respect to the two-dimensional transducer array has been determined in step 930, has corresponding depth and transverse coordinates in the transverse cross-section view.

In step 940, the transverse cross-section view is displayed along with a probe indicator at the depth coordinate and transverse coordinate corresponding to the probe tip. The probe indicator may comprise any appropriate software-generated special effect, allowing the real-time visualization of the probe tip position with respect to the transverse cross-section view of the target zone at the target location.

In step 945, a topographic rendition of the target tissue is generated and displayed. As described herein, data from at least a portion of the transducer arrays may be used map the target tissue based on one or more of a shape of the tissue, the density of the tissue, the relative position of the tissue with respect to the array, the pulsatility of the tissue, or the echogenicity of the tissue.

In step 950, tissue structures within the target tissue are identified on the displayed image of the target tissue. As described herein, the processor coupled to the transducer arrays may be configured to recognize one or more structures or organs of the tissue, such as a blood vessel, a vein, an artery, or tissue masses. The identified tissue structures may be indicated on the displayed image with text labels, different colors, and the like.

Although the above steps show the method 900 for tracking a probe within a target zone in accordance with many embodiments, a person of ordinary skill in the art will recognize many variations based on the teachings described herein. The steps may be completed in a different order. Steps may be added or deleted. Some of the steps may comprise sub-steps. Many of the steps may be repeated as often as beneficial to the measurement(s).

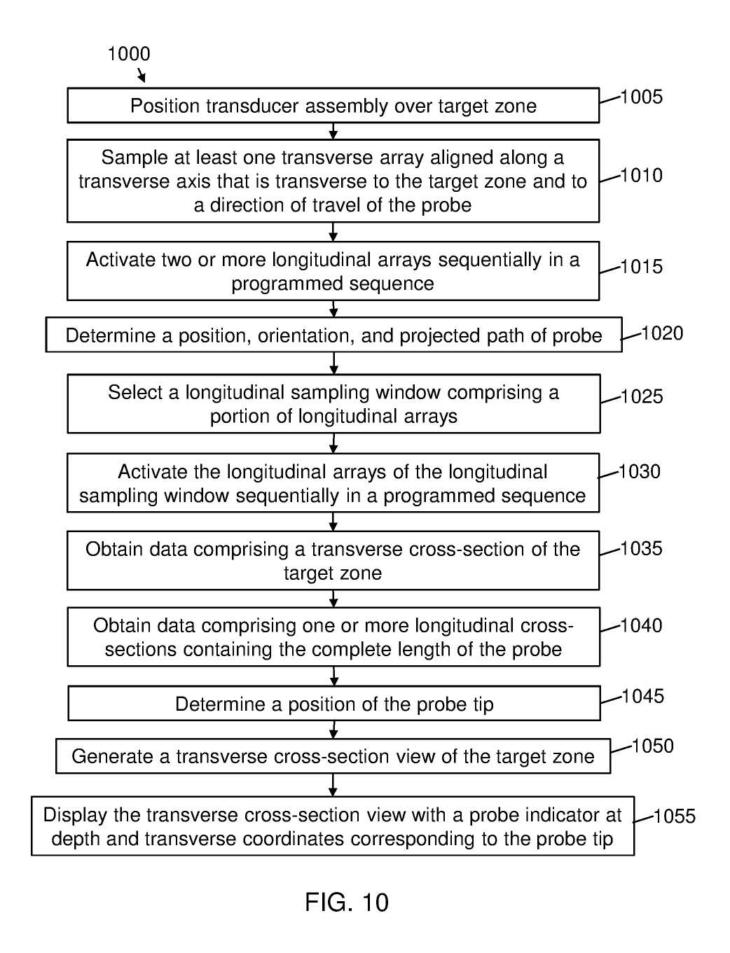

FIG. 10 shows a flowchart of a method 1000 for providing real-time monitoring of a probe at a target zone. Aspects of the some of the steps of method 1000 may be substantially similar to some of the steps of method 900, described with reference to FIG. 9.

At step 1005, the transducer assembly may be positioned over the target zone.

At step 1010, at least one transverse array may be activated, wherein the transverse array extends along a transverse axis that is transverse to the target zone and to a direction of travel of the probe.

At step 1015, two or more longitudinal arrays may be activated sequentially in a programmed sequence, wherein each longitudinal array extends along a longitudinal axis that is transverse to the transverse axis.

At step 1020, a position, orientation, and projected path of the probe is determined based on the data generated with the longitudinal arrays.

At step 1025, a longitudinal sampling window is selected, the longitudinal sampling window comprising a portion of the plurality of longitudinal arrays of the two-dimensional array. As described herein, the longitudinal sampling window may comprise adjacent longitudinal arrays whose corresponding longitudinal scans collectively contain the complete length of the probe.

At step 1030, the longitudinal arrays of the longitudinal sampling window are activated sequentially in a programmed sequence to sample the length of the probe.

At step 1035, data comprising a transverse cross-section of the target zone is obtained from the at least one transverse array.

At step 1040, data comprising one or more longitudinal cross-sections containing the complete length of the probe is obtained from the longitudinal arrays of the longitudinal sampling window.

At step 1045, the position of the probe tip is determined based on the data from the two or more longitudinal arrays.

At step 1050, a transverse cross-section view of the target zone is generated based on the data from the at least one transverse array.

At step 1055, the transverse cross-section view is displayed along with a probe indicator at the depth coordinate and transverse coordinate corresponding to the probe tip.

Although the above steps show the method 1000 for tracking a probe within a target zone in accordance with many embodiments, a person of ordinary skill in the art will recognize many variations based on the teachings described herein. The steps may be completed in a different order. Steps may be added or deleted. Some of the steps may comprise sub-steps. Many of the steps may be repeated as often as beneficial to the measurement(s). For example, steps 1015-1025 may be repeated at a plurality of time points during the procedure, such that the longitudinal sampling window may be dynamically adjusted as the probe travels towards the target zone.

One or more of the steps of the method 900 or 1000 may be performed with various circuitry, as described herein, for example one or more of the processor, controller, or circuit board described above and herein. Such circuitry may be programmed to provide one or more steps of the method 900 or 1000, and the program may comprise program instructions stored on a computer readable memory or programmed steps of the logic circuitry such as programmable array logic or a field programmable gate array, for example.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus for facilitating intra-tissue inspection of a probe at a target zone, the apparatus comprising:
a transducer assembly comprising a two-dimensional array of transducer elements, the two-dimensional array comprising a plurality of transverse arrays and a plurality of longitudinal arrays, wherein each transverse array extends along a transverse axis of the two-dimensional array, and wherein each longitudinal array extends along a longitudinal axis of the two-dimensional array that is transverse to the transverse axis; and
a processor configured to,
activate at least one transverse array, wherein the at least one transverse array extends along a transverse axis that is transverse to the target zone and to a direction of travel of the probe,
activate two or more longitudinal arrays sequentially in a programmed sequence,
receive, from the at least one transverse array, data comprising a transverse cross-section of the target zone,
receive, from the two or more longitudinal arrays, data comprising a longitudinal cross-section of at least a portion of the probe,
determine, based on the data from the two or more longitudinal arrays, a position of a probe tip of the probe with respect to the two-dimensional array,
generate a transverse cross-section view of the target zone based on the data from the at least one transverse array, the transverse cross-section view having depth coordinates and transverse coordinates, and the probe tip having a corresponding depth coordinate and transverse coordinate in the transverse cross-section view, and
display the transverse cross-section view with a probe indicator at the depth coordinate and transverse coordinate corresponding to the probe tip.

2. An apparatus as in claim 1, wherein the processor is further configured to select a longitudinal sampling window comprising a subset of the plurality of longitudinal arrays of the two-dimensional array, the subset comprising one or more longitudinal arrays collectively configured to produce one or more longitudinal cross-sections of a complete length of the probe, and wherein the processor is configured to selectively activate the one or more longitudinal arrays of the longitudinal sampling window.

3. An apparatus as in claim 2, wherein the processor is further configured to adjust a width of the longitudinal sampling window or selection of the subset of the plurality of longitudinal arrays comprising the longitudinal sampling window based on a position or orientation of the probe.

4. An apparatus as in claim 1, wherein the processor is further configured to select a subset of the plurality of longitudinal arrays of the two-dimensional array for use in determination of the position of the probe tip, the subset comprising one or more longitudinal arrays collectively configured to produce one or more longitudinal cross-sections of a complete length of the probe.

5. An apparatus as in claim 1, wherein the two-dimensional array further comprises one or more diagonal arrays extending along a diagonal axis that is oriented at an oblique angle to the transverse axis, and wherein the processor is further configured to activate the one or more diagonal arrays and receive from the one or more diagonal arrays data comprising a diagonal cross-section of at least a portion of the probe.

6. An apparatus as in claim 2, wherein the one or more diagonal arrays comprise two or more diagonal arrays, and wherein the processor is configured to activate the two or more diagonal arrays sequentially in a programmed sequence.

7. An apparatus as in claim 1, wherein the at least one transverse array and the two or more longitudinal arrays are activated simultaneously at similar frequencies.

8. An apparatus as in claim 1, wherein the at least one transverse array and the two or more longitudinal arrays are activated simultaneously at different frequencies.

9. An apparatus as in claim 1, wherein the at least one transverse array and the two or more longitudinal arrays are activated simultaneously at substantially non-interfering frequencies.

10. An apparatus as in claim 1, wherein the two or more longitudinal arrays comprise all of the plurality of longitudinal arrays of the two-dimensional array, and wherein the processor is configured to activate the plurality of longitudinal arrays in a programmed sequence to sample all transducer elements of the two-dimensional array.

11. An apparatus as in claim 1, wherein the processor is configured to determine the position of the probe tip at predetermined time intervals, and update the display of the transverse cross-section of the target zone at each time interval to show the probe indicator at depth and transverse coordinates corresponding to the position of the probe tip determined at each time interval.

12. An apparatus as in claim 11, wherein the predetermined time intervals substantially match a rate of data acquisition by the programmed sequence of the two or more longitudinal arrays.

13. An apparatus as in claim 12, wherein the predetermined time intervals substantially match a rate of data acquisition by each activated transverse array or longitudinal array.

14. An apparatus as in claim 1, wherein all transducer elements of a single activated transverse array or longitudinal array are pulsed simultaneously.

15. An apparatus as in claim 1, wherein transducer elements of a single activated transverse array or longitudinal arrays are each pulsed individually in a timed sequence.

16. An apparatus as in claim 15, wherein the processor is configured to generate a three-dimensional image of the target zone and the probe based on the data received from the at least one transverse array and the two or more longitudinal arrays.

17. An apparatus as in claim 1, wherein the probe indicator comprises one or more symbols or shapes displayed using one or more colors, animations, or other software-generated special effects.

18. An apparatus as in claim 1, wherein the processor is further configured to determine a projected probe path of the probe based on the position of the probe tip at two or more time points, and display the transverse cross-section view with a projected probe trajectory at depth and transverse coordinates corresponding to the projected probe path.

19. An apparatus as in claim 18, wherein one of the two or more time points is an insertion time point of insertion of the probe into the target zone, and wherein the probe tip is at a known, predetermined position at the insertion time point.

20. An apparatus as in claim 18, wherein the projected probe trajectory comprises one or more of a colorized line, dashed line, dotted line, flashing line, or an arrow.

21. An apparatus as in claim 1, wherein the processor is further configured to determine a position, with respect to the two-dimensional array, of a target location within the target zone, and display the transverse cross-section view with a target hit indicator at depth and transverse coordinates corresponding to the probe tip when the position of the target location matches the position of the probe tip.

22. An apparatus as in claim 21, wherein the target hit indicator comprises one or more of a radiating or glowing tip of the probe indicator, a flashing tip of the probe indicator, or a color change of a tip of the probe indicator.

23. An apparatus as in claim 1, wherein the processor is further configured to generate and display a topographical rendition of the target zone based on the data from the at least one transverse array or the two or more longitudinal arrays.

24. An apparatus as in claim 1, wherein the processor is further configured to identify one or more tissue structures of the target zone in the displayed transverse cross-section view, wherein the processor is configured to identify the one or more tissue structures based on one or more of a shape, density, relative position, pulsatility, or echogenicity of the one or more tissue structures as determined with the data from the at least one transverse array or the two or more longitudinal arrays.

25. A method for providing real-time monitoring of a probe at a target zone, the method comprising:
positioning a transducer assembly over the target zone, the transducer assembly comprising a two-dimensional array of transducer elements having a plurality of transverse arrays and a plurality of longitudinal arrays,
activating at least one transverse array, wherein the at least one transverse array extends along a transverse axis that is transverse to the target zone and to a direction of travel of the probe,
activating two or more longitudinal arrays sequentially in a programmed sequence, wherein each longitudinal array extends along a longitudinal axis that is transverse to the transverse axis;
obtaining, from the at least one transverse array, data comprising a transverse cross-section of the target zone,
obtaining, from the two or more longitudinal arrays, data comprising a longitudinal cross-section of at least a portion of the probe,
determining, based on the data from the two or more longitudinal arrays, a position of a probe tip of the probe with respect to the two-dimensional array,
generating a transverse cross-section view of the target zone based on the data from the at least one transverse array, the transverse cross-section view having depth coordinates and transverse coordinates, and the probe tip having a corresponding depth coordinate and transverse coordinate in the transverse cross-section view, and
displaying the transverse cross-section view with a probe indicator at the depth coordinate and transverse coordinate corresponding to the probe tip.

26. A method as in claim 25, further comprising selecting a longitudinal sampling window comprising a subset of the plurality of longitudinal arrays of the two-dimensional array, the subset comprising one or more longitudinal arrays collectively configured to produce one or more longitudinal cross-sections of a complete length of the probe, and wherein activating two or more longitudinal arrays comprises selectively activating the one or more longitudinal arrays of the longitudinal sampling window.

27. A method as in claim 26, further comprising adjusting a width of the longitudinal sampling window or selection of the subset of the plurality of longitudinal arrays comprising the longitudinal sampling window based on a position or orientation of the probe.

28. A method as in claim 25, further comprising selecting a subset of the plurality of longitudinal arrays comprising one or more longitudinal arrays collectively configured to produce one or more longitudinal cross-sections of a complete length of the probe, and wherein the determining comprises determining the position of the probe tip based on data from the subset of the plurality of longitudinal arrays.

29. A method as in claim 25, wherein the two-dimensional array further comprises one or more diagonal arrays extending along a diagonal axis that is oriented at an oblique angle to the transverse axis, and wherein the method further comprises activating the one or more diagonal arrays and receiving from the one or more diagonal arrays data comprising a diagonal cross-section of at least a portion of the probe.

30. A method as in claim 26, wherein the one or more diagonal arrays comprise two or more diagonal arrays, and wherein the two or more diagonal arrays are activated sequentially in a programmed sequence.

31. A method as in claim 25, wherein the at least one transverse array and the two or more longitudinal arrays are activated simultaneously at similar frequencies.

32. A method as in claim 25, wherein the at least one transverse array and the two or more longitudinal arrays are activated simultaneously at different frequencies.

33. A method as in claim 25, wherein the at least one transverse array and the two or more longitudinal arrays are activated simultaneously at substantially non-interfering frequencies.

34. A method as in claim 25, wherein the two or more longitudinal arrays comprise all of the plurality of longitudinal arrays of the two-dimensional array, and wherein activating the two or more longitudinal arrays comprises activating the plurality of longitudinal arrays in a programmed sequence to sample all transducer elements of the two-dimensional array.

35. A method as in claim 25, wherein the determining comprises determining the position of the probe tip is at predetermined time intervals, and the displaying comprises updating the transverse cross-section of the target zone at each time interval to show the probe indicator at depth and transverse coordinates corresponding to the position of the probe tip determined at each time interval.

36. A method as in claim 35, wherein the predetermined time intervals substantially match a rate of data acquisition by the programmed sequence of the two or more longitudinal arrays.

37. A method as in claim 35, wherein the predetermined time intervals substantially match a rate of data acquisition by each activated transverse array or longitudinal array.

38. A method as in claim 25, wherein all transducer elements of a single activated transverse array or longitudinal array are pulsed simultaneously.

39. A method as in claim 25, wherein transducer elements of a single activated transverse array or longitudinal arrays are each pulsed individually in a timed sequence.

40. A method as in claim 39, further comprising generating and displaying a three-dimensional image of the target zone and the probe based on the data received from the at least one transverse array and the two or more longitudinal arrays.

41. A method as in claim 25, wherein, the probe indicator comprises one or more symbols or shapes displayed using one or more colors, animations, or other software-generated special effects.

42. A method as in claim 25, further comprising determining a projected probe path of the probe based on the position of the probe tip at two or more time points, and displaying the transverse cross-section view with a projected probe trajectory at depth and transverse coordinates corresponding to the projected probe path.

43. A method as in claim 42, wherein one of the two or more time points is an insertion time point of insertion of the probe into the target zone, and wherein the probe tip is at a known, predetermined position at the insertion time point.

44. A method as in claim 42, wherein the projected probe trajectory comprises one or more of a colorized line, dashed line, dotted line, flashing line, or an arrow.

45. A method as in claim 25, further comprising determining a position, with respect to the two-dimensional array, of a target location within the target zone, and displaying the transverse cross-section view with a target hit indicator at depth and transverse coordinates corresponding to the probe tip when the position of the target location matches the position of the probe tip.

46. A method as in claim 45, wherein the target hit indicator comprises one or more of a radiating or glowing tip of the probe indicator, a flashing tip of the probe indicator, or a color change of a tip of the probe indicator.

47. An apparatus for facilitating intra-tissue inspection of a probe at a target zone, the apparatus comprising:
transducer assembly comprising a two-dimensional array of transducer elements, the two-dimensional array comprising a plurality of longitudinal arrays; and
a processor configured to,
activate the plurality of longitudinal arrays sequentially in a programmed sequence,
receive, from the plurality of longitudinal arrays, data comprising a plurality of longitudinal cross-sections of the probe,
determine, based on the data from the plurality of longitudinal arrays, a position and an orientation of the probe with respect to the two-dimensional array,
select, based on the position and orientation of the probe, a longitudinal sampling window comprising a subset of the plurality of longitudinal arrays, the subset comprising one or more longitudinal arrays collectively configured to produce one or more longitudinal cross-sections of the probe over a complete length of the probe, and
activate the one or more longitudinal arrays of the longitudinal sampling window sequentially in a programmed sequence.

48. An apparatus as in claim 47, wherein the processor is further configured to adjust a width of the longitudinal sampling window or selection of the subset of the plurality of longitudinal arrays of the longitudinal sampling window, based on a position and orientation of the probe.

49. An apparatus or a method as in any one of the preceding claims, wherein the processor is configured with instructions to define the window so as to correspond to a first area of the array with dimensions sized smaller than a second area of the two-dimensional array and wherein circuitry coupled to the array is configured to sample data over the second area sized larger than the first area.

50. An apparatus or a method as in any one of the preceding claims, wherein the window corresponds to a portion of the array and wherein the processor is configured with instructions to sample in hardware only a portion of the array defined with the window.
